(19) United States
(12) Patent Application Publication
(10) Pub. No.: US 2006/0015137 A1
(43) Pub. Date: Jan. 19, 2006

(54) Retrievable intravascular filter with bendable anchoring members

(76) Inventors: Joel M. WasDyke, Eden Prairie, MN (US); Anthony Vrba, Maple Grove, MN (US)

Correspondence Address: CROMPTON, SEAGER & TUFTE, LLC, 1221 NICOLLET AVENUE, SUITE 800, MINNEAPOLIS, MN, 55403-2420, US

(21) Appl. No.: 10/894,382

(22) Filed: Jul. 19, 2004

(51) Int. Cl. A61M 29/00

(52) U.S. Cl. 606/200

(57) ABSTRACT

Retrievable intravascular filters for insertion within a blood vessel are disclosed. A retrievable intravascular filter in accordance with an illustrative embodiment of the present invention includes an apical head operatively coupled to a number of elongated filter legs that can be used to collect blood clots contained within a blood vessel. A bendable anchoring member coupled to or formed integrally with one or more of the elongated filter legs can be used to temporarily or permanently secure the intravascular filter to the wall of the blood vessel.

### FIELD OF THE INVENTION

The present invention relates generally to devices for filtering blood clots within a blood vessel. More specifically, the present invention pertains to retrievable intravascular filter devices and associated methods for retrieving such devices within the body.

### BACKGROUND OF THE INVENTION

Intravascular filters are used in combination with other thrombolytic agents to treat pulmonary embolism occurring within a patient. Such devices are generally inserted intravenously into a target location of the body (e.g. an artery or vein), and function by capturing blood clots (emboli) contained in the blood stream before they can reach the heart and/or lungs and cause permanent damage to the body. In the treatment of Deep Vein Thrombosis (DVT), for example, such filters can be placed in the vena cava to prevent further blood clotting in the large veins of the lower body. Placement of the filter is typically accomplished percutaneously via the femoral arteries or the jugular vein using a local anesthetic, or by performing a laparotomy with the patient under general anesthesia.

In certain procedures, an introducer sheath may be used to deliver the intravascular filter through the body. Such introducer sheaths are typically tubular in shape and include an internal lumen configured to transport the intravascular filter in a collapsed position through the body. Once advanced to a desired location within the vasculature (e.g. the inferior vena cava), the intravascular filter can then be removed from within the introducer sheath, allowing the device to spring open and engage the vessel wall. A needle, hook, barb, prong, wedge or other attachment means can be used to secure the intravascular filter to the vessel wall.

There are a number of situations in which it may be desirable for a physician to remove the intravascular filter once implanted within the body. In certain circumstances, for example, the risk of pulmonary embolism may be relatively short term (e.g. about two weeks), thus requiring insertion of the device for only a short period of time. Permanent implantation of the intravascular filter in such cases may unnecessarily impede the flow of blood within the vessel, and can lead to further thrombosis growth at the filter implantation site. In other circumstances, it may be desirable to reposition the intravascular filter within the vessel, or to replace the existing filter with a new filter.

### SUMMARY OF THE INVENTION

The present invention relates generally to retrievable intravascular filters for filtering blood clots within the body. Devices and associated methods for retrieving the intravascular filter within a blood vessel are also discussed herein.

A retrievable intravascular filter in accordance with an illustrative embodiment of the present invention may include an apical head operatively coupled to a number of elongated filter legs that can be expanded within a blood vessel to collect blood clots contained in the blood stream. A bendable anchoring member coupled to or formed integrally with one or more of the elongated filter legs can be used to temporarily or permanently secure the filter legs to the inner wall of the blood vessel, thereby preventing the intravascular filter from migrating or tilting within the blood vessel. In certain embodiments, each of the bendable anchoring members can include a coiled member configured to bend from an initially curved shape when attached to the vessel wall to a substantially straight shape for retrieval within a retrieval catheter. In other embodiments, each of the bendable anchoring members can include a spiraled member having a pigtail configuration that can be configured to bend when detached from the vessel wall. A pointed tip portion oriented at an angle relative to the surface of the vessel wall can be used to releasably secure one or more of the filter legs to the vessel wall.

### BRIEF DESCRIPTION OF THE DRAWINGS

FIG. 1 is a perspective view of a retrievable intravascular filter in accordance with an illustrative embodiment of the present invention;

FIG. 2 is a top view showing the retrievable intravascular filter of FIG. 1 disposed along the wall of a blood vessel;

FIG. 3 is an enlarged view showing the anchoring member of one of the elongated filter legs of FIG. 1 in greater detail;

FIG. 4 is an enlarged view showing another illustrative anchoring member including a spiraled member having a pigtail configuration;

FIG. 5 is a partial cross-sectional view showing the retrievable intravascular filter of FIG. 1 temporarily implanted within a blood vessel;

FIG. 6 is a partial cross-sectional view showing a retrieval apparatus advanced to the site of the retrievable intravascular filter of FIG. 5;

FIG. 7 is a partial cross-sectional view showing the detachment of the retrievable intravascular filter using the retrieval apparatus of FIG. 6; and

FIG. 8 is a partial cross-sectional view showing the retrievable intravascular filter of FIG. 5 retracted into retrieval apparatus.

### DETAILED DESCRIPTION OF THE INVENTION

The following description should be read with reference to the drawings, in which like elements in different drawings are numbered in like fashion. The drawings, which are not necessarily to scale, depict selected embodiments and are not intended to limit the scope of the invention. Although examples of construction, dimensions, and materials are illustrated for the various elements, those skilled in the art will recognize that many of the examples provided have suitable alternatives that may be utilized.

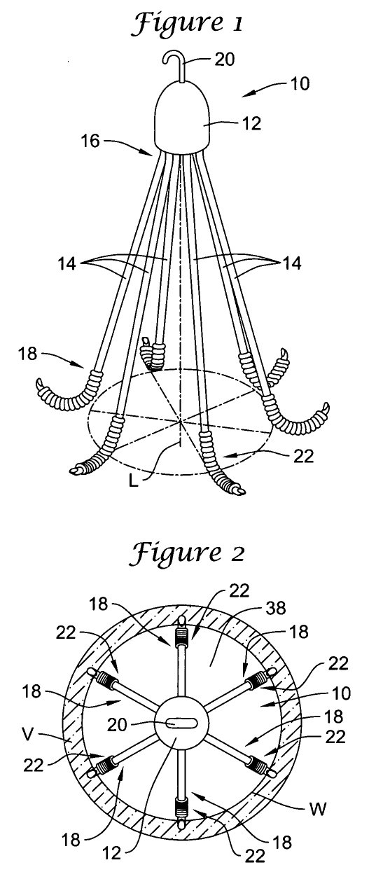

FIG. 1 is a perspective view of a retrievable intravascular filter 10 in accordance with an illustrative embodiment of the present invention. Intravascular filter 10, illustratively a vena cava filter, includes an apical head 12 operatively coupled to a number of elongated filter legs 14 each having a proximal section 16 and a distal section 18. Each of the filter legs 14 may be configured identically with respect to each other, and may be symmetrically spaced about a central longitudinal axis L in a generally conical-shaped configuration when expanded. The filter legs 14 may be collectively arranged about the longitudinal axis L such that the proximal section 16 of each filter leg 14 converges at the apical head 12 to form an apex. In certain embodiments, the filter legs 14 can be biased to expand from a substantially straight position when radially constrained within a catheter or introducer sheath to an outswept position when deployed in a blood vessel.

The filter legs 14 can be formed from a wire, rod, tubing or other elongated member that can be cut and processed to form the general structure of FIG. 1. The dimensions of the filter legs 14 can vary depending on the particular location within the body in which the device is to be implanted. In applications involving the inferior vena cava, for example, the filter legs 14 can be dimensioned to collectively expand to a diameter of about 18 to 32 mm, which is the normal range for the human inferior vena cava. The dimensions of the filter legs 14 can vary, however, allowing the intravascular filter 10 to be implanted in other locations within the body such as the coronary arteries or the peripheral vasculature.

The filter legs 14 can be formed from a metal such as platinum, gold, tantalum, tungsten, titanium, or a metal alloy such as stainless steel (e.g. type 316L), Beta III Titanium, cobalt-chrome alloy, Elgiloy, L605, MP35N, Ta-10W, 17-4PH, or Aeromet 100. In certain embodiments, the filter legs 14 can be formed from a shape-memory material such as nickel-titanium alloy (Nitinol). A slight outward bend can be imparted to each filter leg 14 by heating the alloy beyond its final austenitic temperature, and then bending each filter leg 14 to a pre-defined shape. The filter legs 14 can be configured to revert to their pre-defined (i.e. bent) shape at or near body temperature (37° C.), allowing each individual filter leg 14 to maintain a straight position until deployed within the blood vessel.

A retrieval member 20 coupled to the apical head 12 can be provided to facilitate retrieval of the intravascular filter 10 from the body. The retrieval member 20 can include a hook, loop, clip, or other suitable fastening mechanism that can be used in conjunction with an optional retrieval device to retrieve the intravascular filter 10 from within the blood vessel, if desired. In certain embodiments, the retrieval member 20 can also be used to center the apical head 12 during deployment within the blood vessel to prevent the intravascular filter 10 from becoming off-centered or tilted.

The distal section 18 of one or more of the filter legs 14 can include a bendable anchoring member 22 that can be used to releasably secure the intravascular filter 10 to the wall of the blood vessel. The anchoring member 22 can be formed integral with or as a separate element from the wire, rod, tubing, etc. forming the filter legs 14. In the illustrative embodiment of FIG. 1, for example, the anchoring members 22 are formed as separate members coupled to the distal section 18 of the filter leg 14 by adhesive, welding, crimping, or other suitable attachment method.

FIG. 2 is a top view showing the intravascular filter 10 of FIG. 1 implanted within a blood vessel V. As can be seen in FIG. 2, the filter legs 14 can be configured to extend outwardly from the apical head 12 during deployment to anchor the intravascular filter 10 along the inner wall W of the blood vessel V. The filter legs 14 can be arranged at equidistant intervals such that the filter legs 14 are symmetrically spaced about the longitudinal axis formed by the apical head 12. In the illustrative embodiment of FIGS. 1-2, the intravascular filter 10 is shown having six filter legs 14 arranged at 60° intervals. It should be understood, however, that any number or arrangement of filter legs could be employed, as desired.

When expanded within the blood vessel V, each anchoring member 22 can be configured to pierce the inner wall W of the vessel V as a result of the outwardly directed force exerted by the filter legs 14. The amount of force exerted against the inner wall W can be made sufficient to prevent migration of the intravascular filter 10 within the vessel V without distending the blood vessel V. By altering various design factors such as the dimensions, material composition, and orientation of the filter legs 14, the intravascular filter 10 can be configured to operate in a wide range of locations within the vasculature.

During implantation within the blood vessel V, the filter legs 14 provide a surface upon which blood clots (emboli) can be collected. To facilitate lysing of the collected blood clots, all or a portion of the intravascular filter 10 can include an anti-thrombogenic coating such as herapin (or its derivatives), urokinase, or PPack (dextrophenylalanine proline arginine chloromethylketone) to prevent insertion site thrombosis from occurring. An anti-inflammatory agent such as dexamethasone, prednisolone, corticosterone, budesonide, estrogen, sulfasalazine, mesalamine, or any suitable combination or mixture thereof can also be applied to all or a portion of the intravascular filter 10 to prevent inflammation caused by the engagement of the intravascular filter 10 along the vessel wall W. To prevent the further formation of blood clots within the blood vessel V, an anti-coagulant agent may also be delivered to the site of the intravascular filter 10.

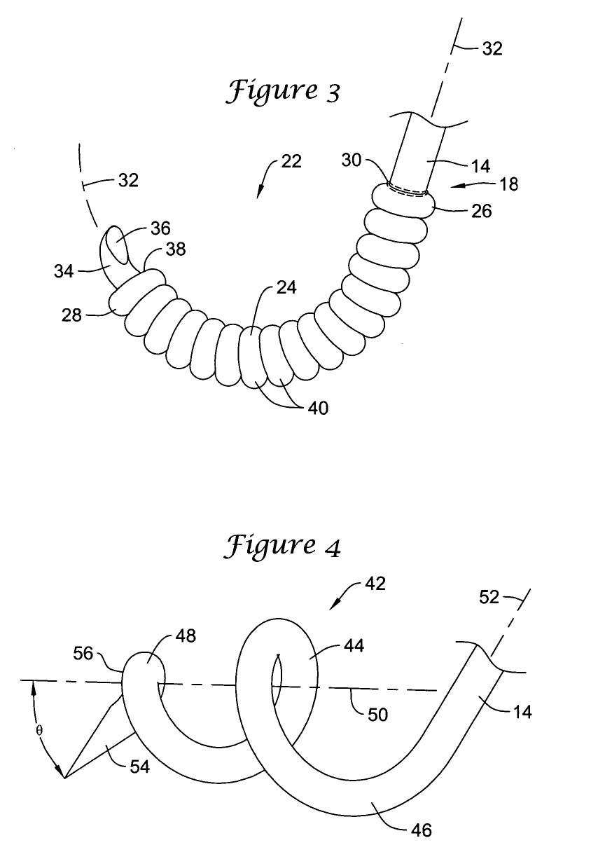

FIG. 3 is an enlarged view showing the anchoring member 22 of one of the elongated filter legs 14 of FIG. 1 in greater detail. As shown in FIG. 3, each anchoring member 22 can include a coiled member 24 having a first end 26 coupled the distal section 18 of the filter leg 14, and a second end 28 that is adapted to rest immediately adjacent to the inner wall of the blood vessel. A solder joint 30 or other suitable attachment means can be provided to secure the first end 26 of the coiled member 24 to the filter leg 14. In an alternative embodiment, the coiled member 24 can be formed integrally with the distal section 18 of the filter leg 14, obviating the need for a separate solder joint 30.

The coiled member 24 can be formed from a spring coil wrapped about an imaginary arc 32 that curves upwardly in a direction towards the apex of the intravascular filter at or near the second end 28 thereof. The general shape of the anchoring member 22 can be formed by taking an elongated piece of wire or tubing, and then wrapping the wire or tubing about a curved mandrel having a shape approximating the imaginary arc 32 depicted in FIG. 3. In one illustrative embodiment, the anchoring member 22 can be formed from a piece of wire or tubing formed from superelastic and/or shape memory material such as nickel-titanium alloy (Nitinol) having a greater flexibility than the material forming the proximal and distal sections 16,18 of the filter legs 14. The generally curved shape of the anchoring member 22 can be formed by wrapping the anchoring member 22 about a curved mandrel having a desired shape, heating the alloy beyond its final austenitic temperature to heat-set the shape, and then allowing the material to cool and revert back to its martensitic state. In use, the alloy can be configured to revert to its pre-defined (i.e. curved) shape at or near body temperature, allowing the anchoring member 22 to maintain a substantially straight position when loaded into the retrieval device.

A pointed tip portion 34 of each anchoring member 22 can be configured to pierce and secure the intravascular filter 10 to the inner wall of the blood vessel. The pointed tip portion 34 may be formed from an unraveled portion of the coiled member 24 that is ground down at section 36 to form a needle or other sharp edge for piercing the inner wall of the blood vessel. Alternatively, the pointed tip portion 34 of each anchoring member 22 can be formed by attaching a separate member such as a needle or barb to the second end 28 of the coiled member 24. In certain embodiments, the pointed tip portion 34 of each anchoring member 22 can be configured to curve upwardly in the general direction of the imaginary arc 32 and at a slight angle relative to the inner surface of the vessel wall to facilitate removal.

During deployment within the blood vessel, the outwardly directed force exerted by the filter legs 14 causes the pointed tip portion 34 of each anchoring member 22 to pierce and secure to the inner wall of the blood vessel. A shoulder 38 formed from the distal-most coil turn of the coiled member 28 can be configured to act as a landing pad for the anchoring member 22, if desired, limiting the engagement depth of the pointed tip portion 34 within the vessel wall.

The structure and material composition of the anchoring members 22 can differ from that of the elongated filter legs 14 to permit the anchoring members 22 to selectively bend when an external force is applied to the intravascular filter 10. In certain embodiments, for example, each anchoring members 22 can be configured to bend from its initially curved shape to a substantially straight shape wherein each anchoring member 22 is aligned with the longitudinal axis of the corresponding filter leg 14. A greater or lesser amount of bendability can be imparted to each anchoring member 22 by varying the size and spacing of the various coil turns 40 forming the coiled member 24, and/or by the selection of materials forming the various components of the intravascular filter 10.

FIG. 4 is an enlarged view showing another illustrative anchoring member 42 including a spiraled member 44 having a pigtail configuration. As shown in FIG. 4, the spiraled member 44 may extend from a first end 46 coupled to or formed integrally with the distal section 18 of the filter leg 14 to a second end 48 that is adapted to rest immediately adjacent to the inner wall of the blood vessel. Similar to the anchoring member 22 of FIG. 3, anchoring member 42 can be configured to selectively bend between a first position engaged along the inner wall of the blood vessel to a second position to facilitate retrieval within a retrieval catheter.

In a first (i.e. deployed) positioned depicted generally in FIG. 4, the spiraled member 44 can be oriented about a spiral axis 50 that is offset from the general longitudinal axis 52 of the filter leg 14. The number of spirals and spacing between each adjacent spiral of the spiraled member 44 can be altered to provide a desired flexibility characteristic within the body. In the illustrative embodiment of FIG. 4, for example, the spiraled member 44 has a pigtail configuration wherein each successive spiral turn decreases in diameter towards the second end 48. It should be understood, however, that the spiraled member 44 could have other configurations, if desired, to alter the flexibility characteristics to the anchoring member 42.

The structure and material composition of the spiraled member 44 can also be altered to impart a greater or lesser amount of flexibility to the anchoring member 42, if desired, allowing the anchoring member 42 to selectively bend when an external force is applied to the intravascular filter 10. In certain embodiments, for example, the spiraled member 44 can be formed from a superelastic and/or shape memory material such as nickel-titanium alloy to permit the anchoring member 42 to easily bend or flex.

As can be further seen in FIG. 4, a pointed tip portion 54 of the spiraled member 44 can be oriented at an angle θ relative to the spiral axis 50. In certain embodiments, the angle θ at which the pointed tip portion 54 departs from the spiral axis 50 can be selected to orient the pointed tip portion 54 at an angle relative to the surface of the vessel wall. A shoulder 56 formed by the distal-most spiral at the second end 48 of the spiraled member 44 can be configured to act as a landing pad for the anchoring member 42, limiting engagement of the pointed tip portion 54 within the vessel wall.

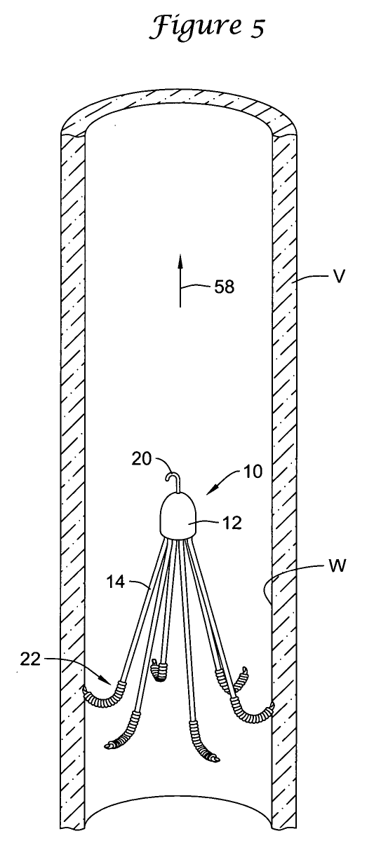

Referring now to FIGS. 5-8, an illustrative method of retrieving the intravascular filter 10 within a blood vessel V will now be described. In a first position illustrated in FIG. 5, intravascular filter 10 is shown in a fully deployed position within a blood vessel V with each anchoring member 22 being temporarily secured to the inner wall W to prevent the movement of the intravascular filter 10 therein. In this position, the filter legs 14 of the intravascular filter 10 can be configured to collect and subsequently lyse blood clots contained in the bloodstream 58.

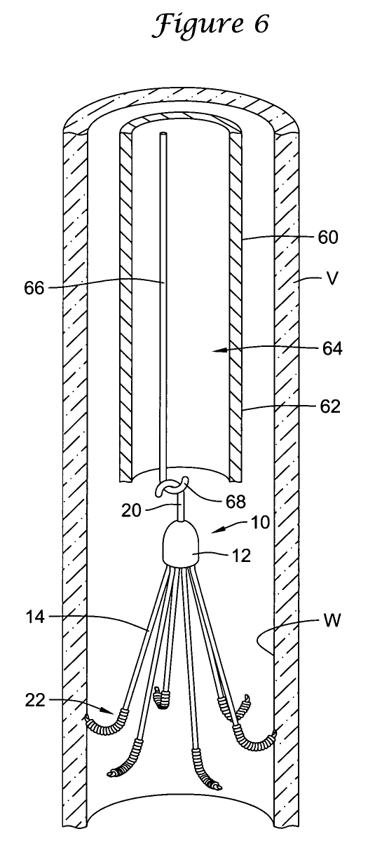

As can be seen in a second position in FIG. 6, a retrieval catheter 60 having a proximal section (not shown), a distal section 62, and an internal lumen 64 configured to collapse and receive the intravascular filter 10 can be advanced to a location adjacent to the apical head 12 of the intravascular filter 10. An elongated member 66 having a hook 68 or other suitable means for engaging the retrieval member 20 can be inserted into the internal lumen 64 of the retrieval catheter 60 and advanced distally beyond the distal section 62 of the retrieval catheter 60. Once advanced to the site of the apical head 12, the elongated member 66 can then be manipulated to secure the hook 68 to the retrieval member 20 on the apical head 12, as shown, for example, in FIG. 6. Once secured thereto, the elongated member 66 can then be pulled proximally while holding the retrieval catheter 60 stationary, causing the anchoring members 22 to detach from the inner wall W of the blood vessel V.

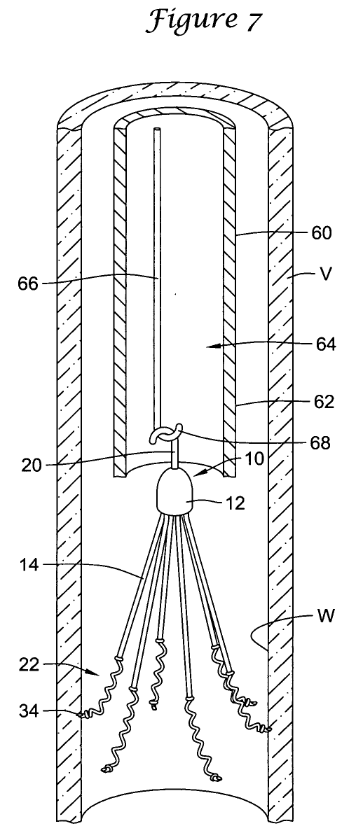

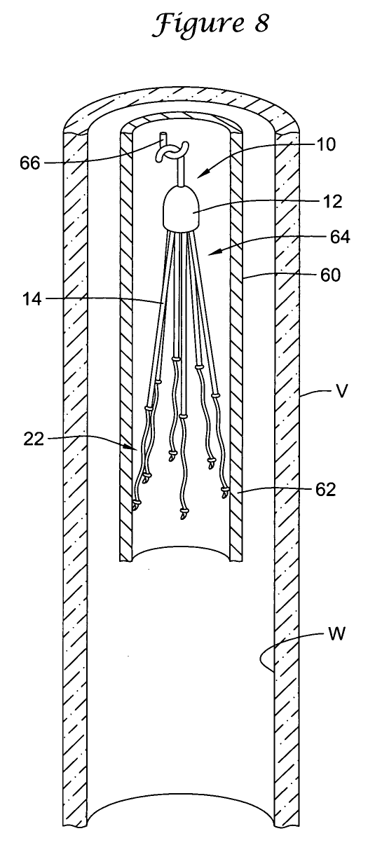

As can be seen in a third position in FIG. 7, each anchoring member 22 can be configured to bend and straighten in a direction substantially parallel with the longitudinal axis of the filter legs 14 as a result of the proximally exerted force applied by the elongated member 66. When this occurs, a small pocket is created at the location where the pointed tip portion 34 engages the inner wall W of the blood vessel V, allowing the anchoring member 22 to be easily detached therefrom. Further retraction of elongated member 66 in the proximal direction causes the filter legs 14 to fold inwardly and collapse within the internal lumen 64 of the retrieval catheter 60, as shown in a fourth position depicted in FIG. 8. Once retracted within the internal lumen 64, the retrieval catheter 60, elongated member 66 and collapsed intravascular filter 10 can then be removed from the body, if desired.

Having thus described the several embodiments of the present invention, those of skill in the art will readily appreciate that other embodiments may be made and used which fall within the scope of the claims attached hereto. Numerous advantages of the invention covered by this document have been set forth in the foregoing description. It will be understood that this disclosure is, in many respects, only illustrative. Changes may be made in details, particularly in matters of shape, size and arrangement of parts without exceeding the scope of the invention.

What is claimed is:

1. A retrievable intravascular filter, comprising:
an apical head;
a plurality of elongated filter legs actuatable between a collapsed position and an expanded position within a blood vessel; and
one or more bendable anchoring members configured to releasably secure the elongated filter legs to the wall of the blood vessel, each of said one or more anchoring members including a coiled member being configured to selectively bend in response to an external force applied thereto.

2. The intravascular filter of claim 1, wherein each anchoring member includes a pointed tip portion.

3. The intravascular filter of claim 2, wherein the pointed tip portion of each anchoring member is configured to engage the wall of the blood vessel at an angle.

4. The intravascular filter of claim 1, wherein each anchoring member is formed from a shape-memory material.

5. The intravascular filter of claim 1, wherein each anchoring member is formed from a superelastic material.

6. The intravascular filter of claim 1, wherein said coiled member is adapted to bend from a curved shape to a substantially straight shape during retrieval.

7. The intravascular filter of claim 1, wherein each anchoring member includes a spiraled member.

8. The intravascular filter of claim 7, wherein the spiraled member has a pigtail configuration.

9. The intravascular filter of claim 7, wherein each elongated filter leg has a longitudinal axis, and wherein the spiraled member is oriented about a spiral axis disposed at an angle away from said longitudinal axis.

10. The intravascular filter of claim 1, wherein the apical head includes a retrieval member.

11. The intravascular filter of claim 10, wherein said retrieval member includes a hook.

12. A retrievable intravascular filter, comprising:
an apical head;
a plurality of elongated filter legs actuatable between a collapsed position and an expanded position within a blood vessel; and
one or more bendable anchoring members configured to releasably secure the elongated filter legs to the wall of the blood vessel, each of said one or more anchoring members including a coiled member and a pointed tip portion.

13. The intravascular filter of claim 12, wherein the pointed tip portion of each anchoring member is configured to engage the wall of the blood vessel at an angle.

14. The intravascular filter of claim 12, wherein each anchoring member is formed from a shape-memory material.

15. The intravascular filter of claim 12, wherein each anchoring member is formed from a superelastic material.

16. The intravascular filter of claim 12, wherein the coiled member is adapted to bend from a curved shape to a substantially straight shape during retrieval.

17. The intravascular filter of claim 12, wherein the apical head includes a retrieval member.

18. The intravascular filter of claim 17, wherein said retrieval member includes a hook.

19. A retrievable intravascular filter, comprising:
an apical head;
a plurality of elongated filter legs actuatable between a collapsed position and an expanded position within a blood vessel; and
one or more bendable anchoring members configured to releasably secure the elongated filter legs to the wall of the blood vessel, each of said one or more anchoring members including a spiraled member and a pointed tip portion.

20. The intravascular filter of claim 19, wherein the pointed tip portion of each anchoring member is configured to engage the wall of the blood vessel at an angle.

21. The intravascular filter of claim 19, wherein each anchoring member is formed from a shape-memory material.

22. The intravascular filter of claim 19, wherein each anchoring member is formed from a superelastic material.

23. The intravascular filter of claim 19, wherein the spiraled member has a pigtail configuration.

24. The intravascular filter of claim 19, wherein each elongated filter leg has a longitudinal axis, and wherein the spiraled member is oriented about a spiral axis disposed at an angle from said longitudinal axis.

25. The intravascular filter of claim 24, wherein the pointed tip portion of each anchoring member is oriented at an angle relative to the spiral axis.

26. The intravascular filter of claim 19, wherein the apical head includes a retrieval member.

27. The intravascular filter of claim 26, wherein said retrieval member includes a hook.

28. A system for retrieving an intravascular filter implanted along the wall of a blood vessel, comprising:
a retrievable intravascular filter comprising an apical head and a plurality of elongated filter legs, one or more of the elongated filter legs including a bendable anchoring member configured to selectively bend in response to an external force applied thereto;
a retrieval catheter having a proximal section, a distal section, and an internal lumen therethrough; and
an elongated member slidably disposed within the internal lumen of the retrieval catheter, said elongated member including means for engaging a retrieval member on the apical head.

29. The system of claim 28, wherein each anchoring member includes a pointed tip portion configured to engage the wall of the blood vessel at an angle.

30. The system of claim 28, wherein each anchoring member includes a coiled member.

31. The system of claim 30, wherein the coiled member is adapted to bend from a curved shape to a substantially straight shape during retrieval.

32. The system of claim 28, wherein each anchoring member includes a spiraled member having a pigtail configuration.

33. A method for retrieving an intravascular filter within a blood vessel, comprising the steps of:
providing a retrievable intravascular filter implanted along the wall of a blood vessel, the intravascular filter including an apical head and a plurality of elongated filter legs, one or more of the elongated filter legs including a bendable anchoring member configured to selectively bend in response to an external force applied thereto;
providing a retrieval catheter having a proximal section, a distal section, and an internal lumen disposed therethrough;
providing an elongated member within the internal lumen of the retrieval catheter, the elongated member including means for engaging a retrieval member on the apical head;
advancing the retrieval catheter and elongated member to a position within the body adjacent to the implantation site of the retrievable intravascular filter;
coupling the elongated member to the retrieval member; and
pulling the elongated member proximally, causing the one or more bendable anchoring members to selectively bend and detach from the wall of the blood vessel.

34. The method of claim 33, wherein each of the one or more bendable anchoring members includes a coil member, and wherein the step of pulling the elongated member proximally causes the coiled member to bend from an initially curved shape to a substantially straight shape.

35. The method of claim 33, wherein each of the one or more bendable anchoring members includes a spiraled member having a pigtail configuration, and wherein the step of engaging the elongated wire member proximally causes the spiraled member to bend and elongate from an initially spiraled shape to a substantially straight shape.

36. The method of claim 33, further comprising the steps of:
retracting the intravascular filter at least in part within the internal lumen of the retrieval catheter; and
removing the intravascular filter from the body.
